(19) United States
(12) Patent Application Publication
(10) Pub. No.: US 2021/0031035 A1
(43) Pub. Date: Feb. 4, 2021

(54) SYSTEM AND METHOD FOR TREATING AND IDENTIFYING AN ARRHYTHMIA

(71) Applicant: PACESETTER, INC., Sytlmar, CA (US)

(72) Inventor: Jason D. Clark, Gig Harbor, WA (US)

(21) Appl. No.: 16/527,526

(22) Filed: Jul. 31, 2019

(51) Int. Cl. A61N 1/362; A61B 5/0464; A61N 1/39; A61B 5/046; A61B 5/00

(52) U.S. Cl. CPC A61N 1/3627; A61B 5/0464; A61N 1/3956; A61B 5/0456; A61N 1/3622; A61B 5/686; A61B 5/046

(57) ABSTRACT

An implantable medical device (IMD) for treating an arrhythmia and a method of treating an arrhythmia with an IMD are provided. The IMD includes electrodes that are configured to be located proximate to heart tissue of interest. At least a portion of the electrodes are configured to deliver therapy. At least a portion of the electrodes are configured to sense cardiac activity (CA) signals. The one more processors, when executing program instructions, are configured to deliver antitachycardia pacing (ATP) therapy through the electrodes to the heart tissue of interest in connection with an arrhythmia and obtain ATP derived CA signals responsive to delivery of the ATP therapy. The IMD determines a characteristic of interest (COI) from the ATP derived CA signals, calculates a probability of an arrhythmia type based on the COI and records the probability of the arrhythmia type.

### BACKGROUND

Embodiments of the present disclosure generally relate to a system and method for treating an arrhythmia.

Tachycardia, or tachyarrhythmia, is a type of arrhythmia that occurs when a heart rate of an individual significantly exceeds the resting heart rate of the individual. A normal heart rate is typically in a range between 60-100 beats per minute, with a heart rate above 100 beats per minute considered a tachycardia.

For individuals utilizing an implantable device, such as a pace maker, implantable cardioverter defibrillators (ICDs), cardiac resynchronization therapy device (CRT-D), or the like, anti-tachycardia pacing (ATP) may be utilized in response to a detected tachycardia in an attempt to reduce the heart rate of the individual back to a normal level. While, ATP works well for terminating ventricular tachycardia (VT), if the arrhythmia is a supra ventricular tachycardia (SVT) that is inappropriately deemed to be VT by the implantable device, an inappropriate shock follows the failed ATP deliveries. These inappropriate shocks after a failed ATP delivery are not only undesired, the condition causing the tachycardia remains untreated. Thus, improved diagnosis techniques and treatment for tachycardia utilizing an implantable device is desired.

### BRIEF SUMMARY

In accordance with embodiments herein, an implantable medical device (IMD) for treating an arrhythmia is provided. The IMD includes electrodes that are configured to be located proximate to heart tissue of interest. At least a portion of the electrodes are configured to deliver therapy. At least a portion of the electrodes are configured to sense cardiac activity (CA) signals. The one more processors, when executing program instructions, are configured to deliver antitachycardia pacing (ATP) therapy through the electrodes to the heart tissue of interest in connection with an arrhythmia and obtain ATP derived CA signals responsive to delivery of the ATP therapy. The IMD determines a characteristic of interest (COI) from the ATP derived CA signals, calculates a probability of an arrhythmia type based on the COI and records the probability of the arrhythmia type.

Optionally, the ATP derived CA signals may be indicative of a response of the heart tissue of interest to the ATP therapy. The COI may include at least one of atrial cycle length, ventricular cycle length, or ventricular-atrial (VA) interval. The ATP derived CA signals may be obtained during at least a beginning or end of delivering the ATP therapy. The probability may be calculated by comparing the COI detected to historical data of prior COI. The arrythmia type may be one of a terminating ventricular tachycardia (VT), or a supra ventricular tachycardia (SVT). The calculating the probability may include determining that the arrythmia type is a supra ventricular tachycardia (SVT), and calculating the probability of a type of SVT based on the COI. The type of SVT may be at least one of Atrio-Ventricular Nodal Reentrant Tachycardia (AVNRT), Atrio-Ventricular Reentrant Tachycardia (AVRT), Atrial Tachycardia (AT), Atrial Rutter (AFL), or Atrial Fibrillation (AF).

Optionally, the one more processors, when executing program instructions, may be configured to transmit a signal including the probability of the arrhythmia type to an external device. The one more processors, when executing program instructions, may be configured to vary pacing of the IMD in response to calculating the probability of the arrythmia type based on the COI. The one more processors, when executing program instructions, may be configured to transmit a signed to a remote device related to scheduling a procedure. The IMD may be a leadless pacemaker.

In accordance with embodiments herein, a method of treating an arrhythmia with an IMD is provided. The method delivers antitachycardia pacing (ATP) therapy through electrodes of the IMD to heart tissue of interest in response to an arrhythmia. The method obtains ATP derived CA signals responsive to delivery of the ATP therapy and determines a characteristic of interest (COI) from the ATP derived CA signals. The method calculates a probability of an arrhythmia type based on the COI and records the probability of the arrhythmia type.

Optionally, the ATP derived CA signals may be indicative of a response of the heart tissue of interest to the ATP therapy. The COI may include at least one of atrial cycle length, ventricular cycle length, or ventricular-atrial (VA) interval. The ATP derived CA signals may be obtained during at least a beginning or end of delivering the ATP therapy. The probability may be calculated by comparing the COI to historical data of prior COI. The calculating the probability may include determining that the arrythmia type is a supra ventricular tachycardia (SVT) and calculating the probability of a type of SVT based on the COI. The method may transmit a signal including the probability of the arrhythmia type calculated to an external device. The method may vary pacing of the IMD in response to calculating the probability of the arrythmia type based on the COI.

### BRIEF DESCRIPTION OF THE DRAWINGS

FIG. 1 illustrates an IMD and external device coupled to a heart in a patient and implemented in accordance with embodiments herein.

FIG. 2 shows a block diagram of an exemplary IMD that is configured to be implanted into the patient in accordance with embodiments herein.

FIG. 3 illustrates a graphical representation of a heart with an implantable medical system located therein in connection with providing pacing therapy, cardiac rhythm management (CRP) therapy and the like.

FIG. 4 illustrates a simple block diagram of at least a portion of the circuitry within an LIMD or SIMD in accordance with an embodiment herein.

FIG. 5 illustrates a block flow diagram of a method of treating an arrhythmia in accordance with embodiments herein.

FIG. 6 illustrates a distributed processing system in accordance with embodiments herein.

FIG. 7A illustrates a screenshot of a display in accordance with an embodiment herein.

FIG. 7B illustrates a screenshot of a display in accordance with an embodiment herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

The term “obtain” or “obtaining”, as used in connection with data, signals, information and the like, includes at least one of i) accessing memory of an external device or remote server where the data, signals, information, etc. are stored, ii) receiving the data, signals, information, etc. over a wireless communications link between a medical device, such as an implantable medical device (IMD), and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The terms “cardiac activity signal”, “cardiac activity signals”, “CA signal” and “CA signals” (collectively “CA signals”) are used interchangeably throughout to refer to an analog or digital electrical signal recorded by two or more electrodes positioned subcutaneous or cutaneous, where the electrical signals are indicative of cardiac electrical activity. The cardiac activity may be normal/healthy or abnormal/arrhythmic. Non-limiting examples of CA signals include ECG signals collected by cutaneous electrodes, and EGM signals collected by subcutaneous electrodes.

The term “ATP derived CA signals” shall mean CA signals sensed or detected during ATP treatment that is delivered in response to the detection of an arrythmia. The CA signals sensed or detected may include sampled CA signals sensed at the beginning of the ATP treatment, at the end of ATP treatment, or during a time period between the beginning of the ATP treatment and end of the ATP treatment. For example, a CA signal for an individual beat includes a P-wave, QRS complex and T-wave, any one or more of which may be a paced atrial or ventricular event corresponding to a pulse of the ATP therapy. As another example, one or more of the P-wave, QRS complex and/or T-wave may be intrinsic and occur between or after a pulse of the ATP therapy.

The term “ventricular cycle length” refers to an intrinsic period of time of a ventricular event (e.g., R-wave) during a cardiac cycle (e.g., the time from the beginning of a first heart beat to the beginning of a second heart beat).

The term “atrial cycle length” refers to an intrinsic period of time of an atrial event (e.g., P-wave) during a cardiac cycle.

The terms “ventricular-atrial interval” and “VA interval” refer to an intrinsic period of time between a ventricular event (e.g., R-wave electrogram) and the next consecutive atrial event (e.g., P-wave electrogram).

The term “marker” refers to data and/or information identified from CA signals that may be presented as graphical and/or numeric indicia indicative of one or more features within the CA signals and/or indicative of one or more episodes exhibited by the cardiac events. Markers may be superimposed upon CA signals or presented proximate to, and temporally aligned with, CA signals. Non-limiting examples of markers may include R-wave markers, noise markers, activity markers, interval markers, refractory markers, P-wave markers, T-wave markers, PVC markers, sinus rhythm markers, AF markers and other arrhythmia markers. As a further non-limiting example, basic event markers may include “AF entry” to indicate a beginning of an AF event, “in AF” to indicate that AF is ongoing, “AF exit” to indicate that AF has terminated, “T” to indicate a tachycardia beat, “B” to indicate a bradycardia beat, “A” to indicate an asystole beat, “VS” to indicate a sensed ventricular beat “Tachy” to indicate a tachycardia episode, “Brady” to indicate a Bradycardia episode, “Asystole” to indicate an asystole episode, “Patient activated” to indicate a patient activated episode. An activity marker may indicate activity detected by activity sensor during the CA signal. Noise markers may indicate entry/start, ongoing, recovery and exit/stop of noise. Markers may be presented as symbols, dashed lines, numeric values, thickened portions of a waveform, and the like. Markers may represent events, intervals, refractory periods, ICM activity, and other algorithm related activity. For example, interval markers, such as the R-R interval, may include a numeric value indicating the duration of the interval. The AF markers indicate atrial fibrillation rhythmic.

The term “COI” refers to a characteristic of interest within CA signals. Non-limiting examples of features of interest include an R-wave, P-wave, T-wave and isoelectric segments. A characteristic of interest may correspond to a peak of an individual R-wave, an average or median P, R or T-wave peak, atrial cycle length, ventricular cycle length, VA interval, or the like.

The term “VA interval” refers to a ventriculoarterial interval, that is an intrinsic period of time between a ventricular event (e.g., R-wave) and the next consecutive atrial event (e.g., P-wave).

The terms “beat” and “cardiac event” are used interchangeably and refer to both normal or abnormal events.

The terms “normal” and “sinus” are used to refer to events, features, and characteristics of, or appropriate to, a heart's healthy or normal functioning.

The terms “abnormal,” or “arrhythmic” are used to refer to events, features, and characteristics of, or appropriate to, an un-healthy or abnormal functioning of the heart.

Embodiments may be implemented in connection with one or more IMDs. Non-limiting examples of IMDs include one or more of implantable leadless monitoring and/or therapy devices, implantable cardioverter defibrillators (ICDs), cardiac resynchronization therapy device (CRT-D), pacemakers, or alternative implantable medical devices. For example, the IMD may represent a cardiac monitoring device, pacemaker, cardioverter, cardiac rhythm management device, defibrillator, leadless pacemaker and the like. The IMD may include one or more structural and/or functional aspects of the device(s) described in U.S. Pat. No. 9,216,285 “Leadless Implantable Medical Device Having Removable And Fixed Components” and U.S. Pat. No. 8,831,747 “Leadless Neurostimulation Device And Method Including The Same”, which are hereby incorporated by reference. Additionally or alternatively, the IMD may include one or more structural and/or functional aspects of the device(s) described in U.S. Pat. No. 8,391980 “Method And System For Identifying A Potential Lead Failure In An Implantable Medical Device” and U.S. Pat. No. 9,232,485 “System And Method For Selectively Communicating With An Implantable Medical Device”, which are hereby incorporated by reference.

Additionally or alternatively, the IMD may be a subcutaneous IMD that includes one or more structural and/or functional aspects of the device(s) described in U.S. application Ser. No. 15/973,195, titled “Subcutaneous Implantation Medical Device With Multiple Parasternal-Anterior Electrodes” and filed May 7, 2018; U.S. application Ser. No. 15/973,219, titled “Implantable Medical Systems And Methods Including Pulse Generators And Leads” filed May 7, 2018; U.S. application Ser. No. 15/973,249, titled “Single Site Implantation Methods For Medical Devices Having Multiple Leads”, filed May 7, 2018, which are hereby incorporated by reference in their entireties. Further, one or more combinations of IMDs may be utilized from the above incorporated patents and applications in accordance with embodiments herein.

Embodiments may be implemented in connection with one or more sensor, monitor, and/or device that measures electronic signals of the heart. Such sensors, monitors, and/or devices may include one or more structural and/or functional aspects of the device(s) described in U.S. Pat. No. 8,665,086, titled “Physiological Data Acquisition and Management System for Use with an Implanted Wireless Sensor” filed Jan. 4, 2012; U.S. Pat. No. 8264,240, titled “Physical Property Sensor with Active Electronic Circuit and Wireless Power and Data Transmission” filed Jul. 20, 2009; which are hereby incorporated by reference in their entireties.

An implantable device, such as an ICD, CRT-D, pacemaker, or the like is provided that upon detection of a tachycardia detects characteristics of interest (COIs) related to the heart to provide information to a clinician, and to diagnose the tachycardia to prevent undesired treatment by the implantable device. As an example, by utilizing anti-tachycardia pacing (ATP), ventricular signals during ATP, and atrial signals during ATP in an implantable device such as dual chamber ICD or CRTD, a specific type of arrhythmia may be diagnosed, allowing the delivery of treatment to terminate the arrhythmia without delivering a shock. As an example, the likelihood or probability the arrhythmia is a VT, SVT, or other condition is determined and displayed for the clinician based on the detected or determined COIS. Based on the probability of the type of arrythmia determined, ATP treatment is terminated to prevent undesired shocks, entrainment is provided, additional testing is performed, or the like.

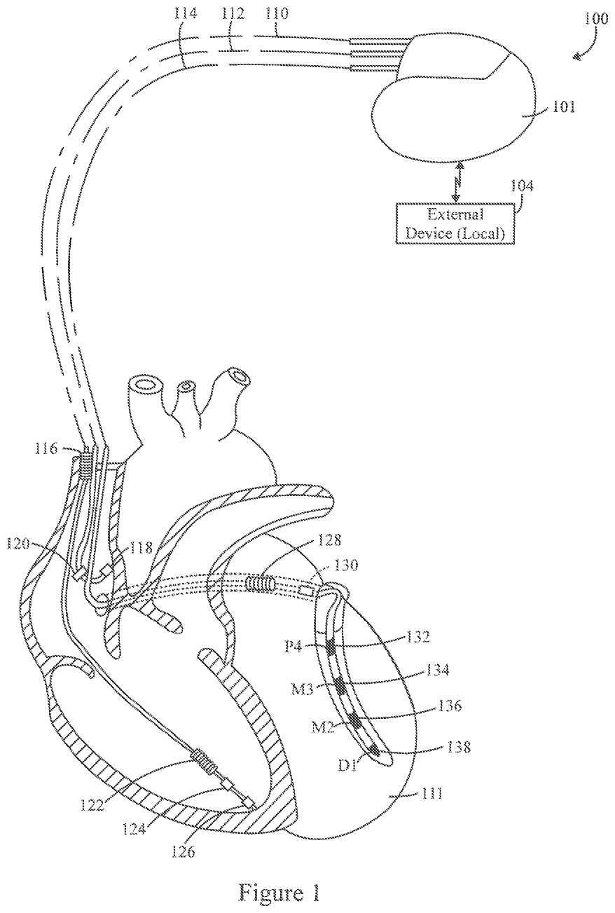

FIG. 1 illustrates an implantable radical device (IMD) 100 and external device 104 coupled to a heart 111 in a patient and implemented in accordance with one embodiment. The external device 104 may be a programmer, an external defibrillator, a workstation, a portable computer, a personal digital assistant, a cell phone, a bedside monitor and the like. The IMD may represent a cardiac monitoring device, pacemaker, cardioverter, cardiac rhythm management device, defibrillator, neurostimulator, leadless monitoring device, leadless pacemaker and the like, implemented in accordance with one embodiment of the present invention. The IMD 100 may be a dual-chamber stimulation device capable of treating both fast and slow arrhythmias with stimulation therapy, including cardioversion, defibrillation, anti-tachycardia pacing and pacing stimulation, as well as capable of detecting heart failure, evaluating its severity, tracking the progression thereof, and controlling the delivery of therapy and warnings in response thereto. The IMD 100 may be controlled to sense atrial and ventricular waveforms of interest, discriminate between two or more ventricular waveforms of interest, deliver stimulus pulses or shocks, and inhibit application of a stimulation pulse to a heart based on the discrimination between the waveforms of interest and the like. Exemplary structures for the IMD 100 are discussed and illustrated in the drawings herewith.

The IMD 100 communicates with a local external device 104. The local external device 104 communicates with a remote server.

The IMD 100 includes a housing 101 that is joined to a header assembly that holds receptacle connectors connected to a right ventricular lead 110, a right atrial lead 112, and a coronary sinus lead 114, respectively. The leads 110, 112, and 114 measure cardiac signals of the heart 111. The right atrial lead 112 includes an atrial tip electrode 118 and an atrial ring electrode 120. The coronary sinus lead 114 includes a left atrial ring electrode 128, a left atrial coil electrode 130 and one or more left ventricular electrodes 132-138 (e.g., also referred to as P1, M1, M2 and D1) to form a multi-pole LV electrode combination. The right ventricular lead 110 includes an RV tip electrode 126, an RV ring electrode 124, an RV coil electrode 122, and an SVC coil electrode 116. The leads 110, 112 and 114 detect IEGM signals that are processed and analyzed as described herein. The leads 110, 112, and 114 also deliver therapies as described herein. In one example, ATP based arrhythmia treatment based on the arrythmia tracking in accordance with embodiments herein is provided. In another example entrainment therapy may be provided, again based on the arrythmia tracking described herein.

During implantation, the external device 104 is connected to one or more of the leads 110, 112 and 114 through temporary inputs. The inputs of the external device 200 receive IEGM signals from the leads 110, 112, and 114 during implantation and display the IEGM signals to the physician on a display. Optionally, the external device 104 may not be directly connected to the leads 110, 112 and 114. Instead, the IEGM cardiac signals sensed by the leads 110, 112 and 114 may be collected by the if 100 and transmitted wirelessly to the external device 104. Hence, the external device 104 receives the IEGM cardiac signals through telemetry circuit inputs. The physician, or another user, controls operation of the external device 104 through a user interface.

During operation, when an arrythmia is detected, the inputs of the external device 104 receive arrythmia based signals providing characteristics of interest (COI) related to the arrythmia. The arrythmia based signals are sensed by the leads 110, 112, and 114, and the COIs are determined and displayed on a display of the external device 104 in accordance with the arrythmia tracking and diagnosis as described herein. Specifically, as will be described in detail herein, in response to detecting an arrythmia, a probability of the type of arrythmia is determined based on the determined COIs and similarly displayed on the display of the external device 104. The determined probability of the type of arrythmia may be made at the IMD 100 or external device 104.

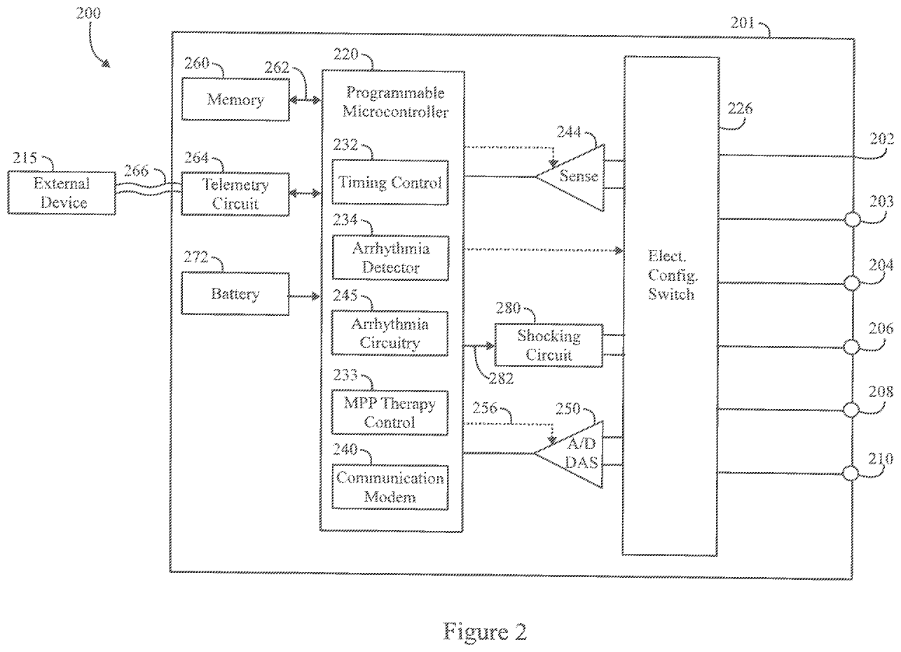

FIG. 2 shows a block diagram of an exemplary IMD 200 that is configured to be implanted into the patient. In one example, the IMD 200 is IMD 100 of FIG. 1. The IMD 200 may treat both fast and slow arrhythmias with stimulation therapy, including cardioversion, pacing stimulation, an implantable cardioverter defibrillator, suspend tachycardia detection, tachyarrhythmia therapy, and/or the like.

The IMD 200 has a housing 201 to hold the electronic/computing components. The housing 201 (which is often referred to as the “can,” “case,” “encasing,” or “case electrode”) may be programmably selected to act as the return electrode for certain stimulus modes. The housing 201 further includes a connector (not shown) with a plurality of terminals 202, 203, 204, 206, 208, 210. The terminals may be connected to electrodes that are located in various locations within and about the heart. The type and location of each electrode may vary. For example, the electrodes may include various combinations of ring, tip, coil, shocking electrodes, and the like.

The IMD 200 includes a programmable microcontroller 220 that controls various operations of the IMD 200, including cardiac monitoring and stimulation therapy. The microcontroller 220 includes a microprocessor (or equivalent control circuitry), one or more processors, RAM and/or ROM memory, logic and timing circuitry, state machine circuitry, and I/O circuitry. The microcontroller also includes an arrythmia detector 234 that is configured to apply one or more arrhythmia detection algorithms for detecting arrhythmia conditions. Specifically, a sensing circuit 244 is selectively coupled to one or more electrodes (e.g., electrodes 132-138 of FIG. 1) that perform sensing operations, through a switch 226 to detect the presence of cardiac activity (CA) in the chamber of the heart 111. Optionally, the IMO 200 may include multiple sensing circuits 244, where each sensing circuit is coupled to one or more electrodes and controlled by the microcontroller 220 to sense electrical activity detected at the corresponding one or more electrodes. The sensing circuit 244 may operate in a unipolar sensing configuration or a bipolar sensing configuration.

When an arrythmia is detected by the arrythmia detector 234, the sensed cardiac activity from the sensing circuit 244 is received by arrythmia circuitry 245 that determines characteristics of interest (COIs) related to the arrythmia and sensed cardiac activity. The arrythmia circuitry 245 may command therapy control circuitry 233 of the microcontroller 220 to provide a predetermined therapy, including ATP, so that the arrythmia circuitry 245 may obtain ATP derived CA signals responsive to the delivery of the ATP. By determining the changes in the COIs as a result of the ATP, the determinations can be utilized by the arrythmia circuitry 245 to determine a probability of the type of arrythmia based on the COIs obtained. The arrythmia circuitry 245 may then record the calculated probability within the memory 260, and/or display the probability on a display for use by a clinician.

In one example, the microcontroller 220 determines the probability of the type of arrythmia by utilizing the COIs in an algorithm. The COIs utilized by the algorithm may include COIs obtained before a treatment, such as ATP, is delivered, COIs obtained at the beginning of ATP treatment, COIs obtained during the ATP treatment, COIs obtained at the end of the ATP treatment, or the like.

In another example, the microprocessor determines the probability of the type of arrythmia based on obtained COIs by comparing the COIs to historical data in a look-up table. Historical data includes arrythmia based COIs from previous patients. The arrythmia based COIs from previous patients may be obtained from a study, health database, paper, or the like. These previous patient COIs may include average COIs, specific COIs, ranges of COIs, or the like. Specifically, in one example, the COIs may include atrial cycle length, ventricular cycle length, the time from the right ventricular signal to the right atrium signal, or ventriculoarterial interval (VA interval), or the like.

Thus, the historical data in the look-up table can include an average length of time for each COI that is indicative of a predetermined arrythmia, including ventricular tachycardia (VT), supra ventricular tachycardia (SVT), specific types of SVT including an Atria-Ventricular Nodal Reentrant Tachycardia (AVNRT), Atrio-Ventricular Reentrant Tachycardia (AVRT), Atrial Tachycardia (AT), Atrial Huller (AFL), or Atrial Fibrillation (AF), or the like. In one example, predetermined probabilities are associated with ranges of times for COIs within the historical data in the look-up table. Therefore, when a detected or determined time period of the obtained COI falls within a specific range of time within the historical data, the predetermined probability that is associated with that range in the look-up table is displayed. More specifically, in one example, the historical data includes a ventricular cycle length range of between 250 ms-300 ms and an associated probability of a high likelihood the arrythmia is a VT. Therefore, if a ventricular cycle length is obtained that is 275 ms, based on the look-up table, a display for the clinician provides the likelihood the arrythmia is a VT is high.

The IMD 200 further includes an analog-to-digital (A/D) data acquisition system (DAS) 250 coupled to one or more electrodes via the switch 226 to sample cardiac signals across any pair of desired electrodes. The A/D converter 250 is configured to acquire intracardiac electrogram signals, convert the raw analog data into digital data and store the digital data for later processing and/or telemetric transmission to an external device 215 (e.g., a programmer, local transceiver, or a diagnostic system analyzer). The A/D converter 250 is controlled by a control signal 256 from the microcontroller 220.

The switch 226 is managed, by the microcontroller 220, to sense the cardiac events at an LV or RV electrode along an LV or RV lead that includes multiple LV or RV electrodes and to enable the burst pacing therapy to be delivered by at least one of the multiple LV or RV electrodes before the HV shock is delivered along at least one of the following shocking vectors: i) superior vena cava coil electrode and RV coil electrode to a CAN electrode, ii) the RV coil electrode to the CAN electrode, or iii) the RV coil electrode to the superior vena cava coil electrode.

The switch 226 may be coupled to an LV lead having multiple LV electrodes, at least one of the LV electrodes configured to be located proximate to the LV site corresponding to the pacing site and to deliver the burst pacing therapy. The switch 226 may be further coupled to a second lead with at least one of a superior vena cava (SVC) coil electrode, or an RV coil electrode, the shock vector including a CAN of the IMD and at least one of the SVC coil electrode or the RV coil electrode.

The microcontroller 220 is operably coupled to a memory 260 by a suitable data/address bus 262. The programmable operating parameters used by the microcontroller 220 are stored in the memory 260 and used to customize the operation of the IMD 200 to suit the needs of a particular patient. The operating parameters of the IMD 200 may be non-invasively programmed into the memory 260 through a telemetry circuit 264 in telemetric communication via communication link 266 (e.g., MICS, Bluetooth low energy, and/or the like) with the external device 215. The memory 260 is also utilized to store COIs of the heart, arrythmia data, historical data, or the like for use in the systems and methodologies herein.

A battery 272 provides operating power to all of the components in the IMD 200. The battery 272 is capable of operating at low current drains for long periods of time, and is capable of providing a high-current pulse (for capacitor charging) when the patient requires a shock pulse (e.g., in excess of 2 A, at voltages above 2 V, for periods of 10 seconds or more). The battery 272 also desirably has a predictable discharge characteristic so that elective replacement time can be detected. As one example, the IMD 200 employs lithium/silver vanadium oxide batteries.

The IMD 200 is further equipped with a communication modem (modulator/demodulator) 240 to enable wireless communication with other devices, implanted devices and/or external devices. In one implementation, the communication modem 240 may use high frequency modulation of a signal transmitted between a pair of electrodes. As one example, the signals may be transmitted in a high frequency range of approximately 10-80 kHz, as such signals travel through the body tissue and fluids without stimulating the heart or being felt by the patient.

The microcontroller 220 further controls a shocking circuit 280 by way of a timing control 232. The shocking circuit 280 generates shocking pulses of low (e.g., up to 0.5 joules), moderate (e.g., 0.5-10 joules), or high energy (e.g., 10 to 40 joules), as controlled by the microcontroller 220. The shocking circuit 280 is controlled by the microcontroller 220 by a control signal 282.

Although not shown, the microcontroller 220 may further include other dedicated circuitry and/or firmware/software components that assist in monitoring various conditions of the patient's heart and managing pacing therapies.

In accordance with embodiments, the IMD 200 may represent a subcutaneous implantable cardioverter defibrillator (S-ICD). The communication modem 240 is configured to wirelessly communicate with a leadless pacemaker, such as to pass timing information there between. The communication modem 240 may transmit timing information to a leadless pacemaker such as when sending an instruction for the leadless pacemaker to deliver a burst pacing therapy. The communication modem 240 may receive timing information from a leadless pacemaker such as when receiving a direction from the leadless pacemaker that a burst pacing therapy has been delivered or is currently being delivered and that S-ICD should now deliver the HV shock.

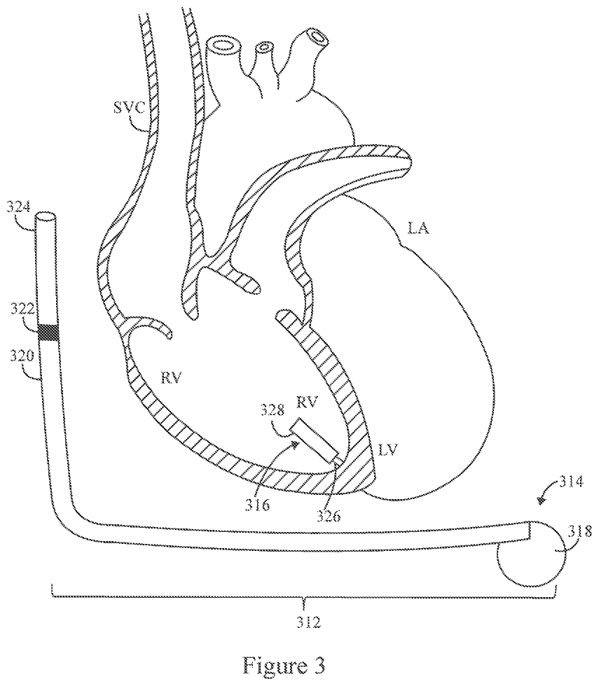

FIG. 3 illustrates a graphical representation of a heart with an alternative implantable medical system 312 to provide ATP based arrhythmia treatment and tracking in accordance with embodiments herein. Additionally, the system 312 provides pacing therapy, cardiac resynchronization therapy (CRT) as well as general arrhythmia therapy. The system 312 includes a first leadless implantable medical devices (LIMD) 316 configured to be implanted entirely within a corresponding first chamber of the heart. The system 312 also includes a subcutaneous implantable medical device (SIMD) 314 configured to be implanted in a subcutaneous area exterior to the heart.

In the example of FIG. 3, the LIMD 316 is implanted in the right ventricle. Optionally, additional LIMDs may be implanted in the left atrium and/or the left ventricle. Alternatively, the LIMD 316 may be implanted in other chambers and/or other positions exterior to the heart. For example, the LIMD 316 may be implanted in the right atrium, left atrium, or left ventricle. Optionally, more than one LIMD 316 may be utilized with each LIMD 316 positioned in a different chamber of the heart. The LIMD 316 is configured to deliver various therapies, including pacing therapy, antitachycardia pacing therapy, and the like.

In the example of FIG. 3, the SIMD 314 is positioned in a subcutaneous area. The subcutaneous implantable cardioverter-defibrillator is a device that does not require insertion of a transvenous lead. Rather, the SIMD includes a subcutaneous pulse generator that may be implanted in the left lateral chest and a subcutaneous left parasternal lead-electrode. Optionally, the SIMD 314 may be positioned in a different subcutaneous area, including proximate to the lower apex of the left ventricle and/or right ventricle. The SIMD 314 is configured to deliver various arrhythmia therapies, including pacing therapy, antitachycardia pacing (ATP) therapy, cardioversion therapy, defibrillation therapy and the like.

In one example, ATP therapy is delivered through electrodes 322, 324 to the heart tissue of interest in connection with an arrhythmia experienced by the heart. Specifically, ATP therapy may be provided specifically to sense ATP derived CA signals responsive to the delivery of the ATP therapy.

The SIMD 314 also includes a housing 318 having a header configured to be connected to a lead 320. The lead 320 includes one or more electrodes 322, 324 positioned along a length thereof. The housing 318 is also configured to operate as an electrode. The electrodes 322, 324 and the housing 318 are configured to perform sensing (along one or more sensing vectors) and to deliver various types of therapy. The lead 320 is positioned such that the electrodes 322 and 324 are positioned proximate to (but outside of) various regions or chambers of the heart. In the example of FIG. 3, the SIMD 314 is positioned proximate to the apex of the LV, while the electrode 322 is positioned at an intermediate point along the LV and the electrode 324 is positioned proximate the LA. Optionally, the SIMD 314 and lead 320 may be positioned in alternative locations and include alternative numbers of electrodes. Optionally, the SIMD 314 may be configured to operate without any lead 320 connected thereto. For example, the housing 318 of the SIMD 314 may include one or more electrically separate electrodes, where one combination of electrodes cooperates cooperate to perform sensing and the same or a different combination of electrodes cooperates to deliver therapy.

The LIMD 316 has a housing 328 with a proximal end 326 that is configured to engage local tissue in the right ventricle. Electrodes (not illustrated in FIG. 3) may be located along the housing 328 at various positions and combinations. The internal electrical components and electrodes may be implemented as described in U.S. Publication No. 2014/0107723, which is expressly incorporated herein by reference in its entirety.

As explained hereafter, the system 312 provides an LIMD that delivers ATP treatment based on the detection of an arrythmia. The SIMD monitors, or tracks the ATP derived CA signals. The SIMD detects the ATP derived CA signals throughout the ATP treatment, including the beginning of treatment and the end of the treatment. In particular, the ATP derived CA signals are indicative of a response of the heart tissue of interest to the ATP therapy. Then based on the ATP derived CA signals, and consequently the response of the heart tissue of interest to the ATP therapy, a determination is made regarding the probability of the type of arrythmia that is presented. Consequently, based on the type of arrythmia presented, treatment, including ATP, entrainment, or the like may be utilized to treat the arrythmia.

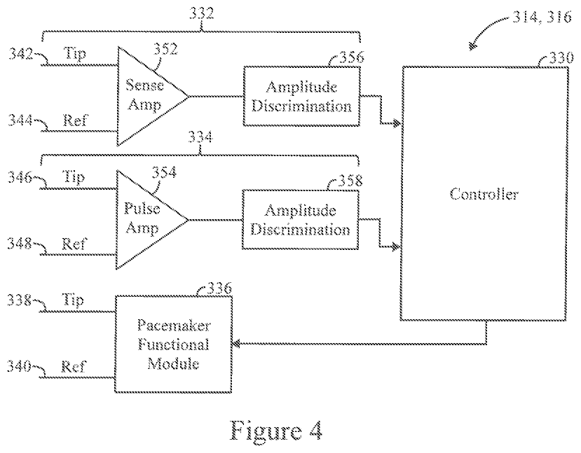

FIG. 4 illustrates a simple block diagram of at least a portion of the circuitry within an LIMD 316 and SIMD 314. The SIMD 314 and LIMD 316 each include a controller 330 that is coupled to cardiac sensing circuitry 332 and pulse sensing circuitry 334. The controller 330 also utilizes or communicates with various other electronic components, firmware, software and the like that generally perform sensing and pacing functions (as generally denoted by a pacemaker functional block 336). The cardiac sensing circuitry 332 is configured to detect intrinsic and paced cardiac events. The pulse sensing circuitry 334 is configured to detect event markers. The sensing circuitry 332 and 334 may be tuned in different manners based upon various characteristics, including whether the sensing circuitry 332, 334 is listening for a near field (NF) or far field (FF) signals and the nature of the signal being sensed (e.g., a far field cardiac signal, a far field event marker, a near field cardiac signal, a near field event marker).

The controller 330 is configured to detect cardiac activity (CA) from the sensing circuitry 332 and 334 that determines characteristics of interest (COIs) related to the arrythmia and senses cardiac activity. The controller 330 may also actuate the electrodes 322, 324 to provide a predetermined therapy, including ATP, to obtain ATP derived CA signals responsive to the delivery of the ATP. By determining the changes in the COs as a result of the ATP therapy, the COIs can be utilized by the controller 330 to calculate a probability of the type of arrythmia based of the COIs detected and record the probability within a memory of the controller 330. Specifically, the controller 330 is configured to function similarly to the microcontroller 220 of FIG. 2 in determining or calculating probabilities of the type of arrythmia, including utilizing look-up tables, algorithms, or the like based on the detected COIs.

Inputs 338-348 are provided to the cardiac and pulse sensing circuitry 332 and 334. By way of example, with reference to LIMD 316, inputs 342 and 344 may be coupled to tip and reference electrodes that supply sensed signals to a sensing amplifier 352. Inputs 346 and 348 may be coupled to the same or different Up and reference electrodes to provide sensed signals to a pulse amplifier 354. An output of the sensing amplifier 352 is supplied to amplitude discriminator 356, while an output of the pulse amplifier 354 is supplied to amplitude discriminator 358. Outputs of the amplitude discriminators 356 and 358 are then provided to the controller 330 for subsequent analysis and appropriate actions. With reference to the SIMD 314, the inputs 342 and 344 may be coupled to various combinations of the electrodes 322, 324 and the electrode formed by the housing 318.

The pulse amplifier 354 and amplitude discriminator 358 are configured to detect select communications pulses having one or more known predetermined formats. For example, the gain of the amp 354 and threshold of the discriminator 358 may be set to pass only signals below a select pulse maximum threshold and/or pulses having a select duration. Optionally, the discriminator 358 may include a band pass, low pass or high pass filter set to only pass pulses within a select frequency range. The amp 354 and/or discriminator 358 may have a high pass frequency (e.g., 500 Hz-10 KHz). The pulse amp 354 and amplitude discriminator 358 may also be configured to sense pacing pulses delivered in the local and/or remote chambers. The communication pulses sensed by the pulse amp 354 and amplitude discriminator 358 represent event markers that are delivered to the controller 330 and used to indicate different events of interest (e.g., physiologic and non-physiologic events or actions).

Therefore, for the IMDs 100, 200, 314, and 316, each has sensing circuitry to detect COIs associated with the heart. Each may also detect an arrythmia, and provide ATP in response to detection of the arrythmia. Each IMD 100, 200, 314, and 316 may also obtain ATP derived CA signals as a result of the ATP and evacuate COIs of the CA signals to determine a probability of the type of arrythmia provided. This probability is then recorded and displayed for use by a clinician in treating, managing, and/or monitoring the arrythmia.

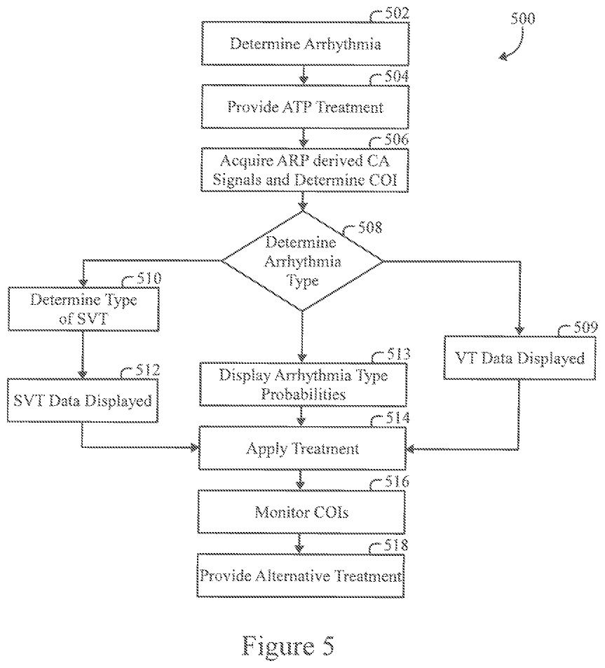

FIG. 5 illustrates a method 500 for treating an arrythmia. In one example, the arrythmia is a tachycardia and an IMD is utilized to treat the arrythmia. In examples, the IMD is any one of IMD 100, IMD 200, SIMD 314, or LIMD 316, described herein. Optionally the EI may be utilized during the treatment in examples includes EI 104, or EI 215.

At 502, an arrythmia is determined by one or more processors of one of the IMD or EI based on at least one CA signal detected by one or more sensors of the IMD, or communicated to the IMD. In one example, the one or more sensors are electrodes of an electrocardiograph machine that monitors the conductive current of the heart over time, and in particular over cycles. The determination of the arrythmia may be stored in the memories of the IMD and/or EI, remote memories or devices, in the cloud, or the like.

At 504, in response to the determination of the arrythmia, the one or more processors of the IMD deliver antitachycardia pacing (ATP) therapy through the electrodes to the heart tissue of interest in connection with the arrhythmia. As a result of the ATP, ATP derived CA signals are generated for evaluation.

At 506, the one or more processors obtain ATP derived CA signals responsive to the ATP therapy. In one example, the ATP derived CA signals are obtained at the beginning of the delivery of the ATP treatment. In another example, the ATP derived CA signals are obtained at the end of the delivering of the ATP treatment. Alternatively, the ATP derived CA signals are obtained both at the beginning and end of the delivery of the ATP treatment. The one or more processors then determine characteristics of interest (COIs) from the ATP derived CA signals. In examples, the COIs include the atrial cycle length (CL), the ventricular CL, and VA interval. Specifically, the atrial CL, ventricular CL, and VA interval may be directly measured, or may be calculated based on detected characteristics or parameters of the heart.

At 508, the one or more processors calculate a probability of an arrhythmia type based on the COI. In one example, the COIs are utilized in association with a model (e.g., an algorithm) in order to make calculations based on the COIs. The models may be used to determine if the arrhythmia is a terminating ventricular tachycardia (VT), supra ventricular tachycardia (SVT), or the like. Specially, the model in one example determines a score based on the COIs. Based on the score, the model determines the type of arrhythmia. The score may be based on a scale between 1-100. Thus, in one example, if the atrial cycle length is 400 ms and the ventricular cycle length is 400 ms, the score provided may be 85 out of 100. The model may use a threshold level such as 80. When the score exceeds the threshold, a SVT is determined to be presented. Alternatively, when the atrial cycle is 400 ms and the ventricular cycle length is 350 ms, the model may assign a score of 70. The model may then determine that the score of 70 is below the threshold level of 80, and the model determines that the episode is an VT.

In yet another example, the model may similarly provide a probability that an episode represents VT or SVT. Thus, again, in an example when a score is provided on a scale of 1-100, when the score is in a range form 100-90, the model may declare a 100% probability that the arrhythmia is a VT episode. Alternatively, if the score is in a range between 80-90, the model may declare an 80% probability that the arrhythmia is a VT episode. Similarly, when the score is in a range between 50-60, the model may indicate an 80% probability exists that the arrhythmia is an SVT. Alternatively, when a score is provided between 70-80, the model may declare a 50% probability of a VT and a 50% probability of an SVT. Thus, the model may utilize COIs to determine or classify an arrhythmia, or may provide an indication of a probability a certain arrhythmia is presented.

Alternatively, the model may be implements by one or more processors comparing the COIs to a look-up table that includes entries associating select combinations of measurements to certain episodes/conditions, namely whether a VT or SVT is indicated. For example, if the atrial cycle length is 400 ms and the ventricular cycle length is 400 ms, and the VA interval is 110 ms, the look-up table may indicate that the three combined COIs are indicative of a SVT episode. Alternatively, when the atrial cycle length is 400 ms, and the ventricular cycle length is 350 ms, a VT may be provided in the look up table.

At 508, if a determination is made that the likelihood, or probability the arrythmia is a VT is above a threshold amount, flow moves 509, and VT data is displayed at 509. In one example, the threshold amount is 1%. In the example, the probability or percentage may be displayed on the screen of an external device. Alternatively or additionally, the probability or percentage may be displayed on the screen with percentages or probabilities of other arrythmias types, including SVT displayed thereon. In yet another example, in addition to percentages or probabilities, one or more of the COIs measured, determined, or calculated at 506 may also be displayed. Alternatively or additionally, while a percentage may be provided, other indicia may be provided. For example, an indicia indicator may provide that the probability of a VT episode is “high”, “medium” or “low”.

Alternatively or additionally, in another example, the threshold amount or value may be 50%, Thus, the probability of 50%-60% likelihood of VT represents a low likelihood, 60%-90% represents a medium likelihood, and 90%-100% represents a high likelihood. In yet another example, a predetermined color may be provided on the screen associated with indicating the probability is within a predetermined range. For example, when the likelihood of a select episode is in a range between 50%-60%, red may be displayed. When the likelihood of a select episode is in a range between 60%-90%, yellow is displayed, and when in a range between 90%-100% green may be displayed.

If at 508, a determination is made that the likelihood, or probability that the arrythmia is an SVT episode is above a threshold amount, flow moves to 510. At 510, additional determinations are made to determine the type of SVT episode is presented. Specifically, in one example the COIs are analyzed by one or more algorithms to diagnose a specific SVT episode, including an Atrio-Ventricular Nodal Reentrant Tachycardia (AVNRT), Atrio-Ventricular Reentrant Tachycardia (AVRT), Atrial Tachycardia (AT), Atrial Rutter (AFL), or Atrial Fibrillation (AF). In another example, the COIs are compared to historical data of related prior COIs in a look-up table to determine the specific SVT. In one example, the look-up table includes sets of COIs and an associated probability of a type of arrythmia associated with each set. For example, if the atrial cycle length is 375 ms, the ventricular cycle length is 375 ms and the VA interval is 200 ms, the associated probability may be an 80% probability of a SVT episode. Alternatively, if the atrial cycle length is 350 ms, the ventricular cycle length is 300 ms, and the VA interval is not consistent beat to beat, the look-up table may indicate a 90% probability that an VT episode is associated with the set of COIs. Alternatively and additionally, while a set of COIs may be determined over a single heart beat cycle, in other examples, average COIs over multiple heart beat cycles are utilized in association with a lookup table model.

In one example, when the COIs indicate that post paced interval (PPI) minus tachycardia cycle length (TCL) is above a first threshold rate of 130 ms, the one or more processors determine the SVT is an AVNRT episode. When the TCL is below a second threshold rate, (e.g., 100 ms), the one or more processors determine the SVT is an AVRT episode. When the TCL is less than the first threshold, but greater than the second threshold, or in an example between 100 ms and 130 ms, a borderline condition is provided where the probabilities of each condition may be provided.

After the type of SVT episode is determined at 510, at 512 SVT data is displayed. In one example, the probability or percentage of a specific SVT is displayed on the screen. Similarly, percentages or probabilities of more than one SVT may also be displayed. Alternatively or additionally, the probability or percentage of one or more SVTs may be displayed on the screen with percentages or probabilities of other arrythmias types, including VT displayed thereon. Alternatively or additionally, no percentage or probability is displayed, and instead the classification or diagnosis is provided, such as “AVRT determined” or similar indicia indicator, or communication on the display to indicate the arrythmia.

The operations at 509 and 510 may occur concurrently with both data related to a VT and SVT appearing on the display simultaneously. Specifically, in one example, the likelihood that an arrythmia is a VT may be 80%, while the likelihood that the arrythmia is an SVT is 15%. Thus, in an example when the threshold probability is 1%, both the 80% VT probability and 15% SVT probability would be displayed on a display, or screen. In this manner, a clinician is provided with additional information related to a classification or diagnosis in determining a treatment accordingly. Thus, if the percentages are 50% VT and 45% SVT, a clinician may desire to take additional measurements before recommending a treatment, or otherwise, may note the probabilities prior to treatment to provide more data to review after treatment.

In yet another example, in addition to percentages or probabilities, one or more of the heart COIs determined at 506 are also displayed. Alternatively, or additionally, while a percentage may be provided, in other examples a score is provided. Specifically, if a score is within a predetermined range, an indicator indicia may provide that the probability of a type of SVT is “high”, while if in other predetermined ranges, “medium” or “low”.

Alternatively or additionally, in another example the threshold amount is 50%. A determination of 50%-60% likelihood of a type of SVT may represent a low likelihood, 60%-90% represent a medium likelihood, and 90%-100% represent a high likelihood. In yet another example, a predetermined color is provided on the screen indicating that the probability is in a predetermined range. For example, when the likelihood is in a range between 50%-60% red may be displayed, when the likelihood is in a range between 60%-90% yellow is displayed, and when in a range between 90%-100% green may be displayed. Similarly, text or indicator indicia, including “leans towards AVNRT/AVRT/AT” may be displayed, again to provide additional information for the clinician.

Displaying a probability may include, as examples, presenting percentages, scores, color, text indicia, or the like. Optionally, a number value and/or percentage does not have to be displayed. Instead, other indicators of probability may be utilized to display the probability, including indicia indicating “leans towards”, “low”, “medium”, and “high”, or the like. Similarly, text or indicator indicia that indicates the diagnosis as a VT, SVT, AVNRT, AVRT, AT, or the like may be displayed, in that the displayed text or information is based on the algorithm, calculations, look-up comparison, or the like.

If at 508, a determination is made that the likelihood, or probability that the arrythmia is a VT is below a VT threshold amount, and a probability that the arrythmia is an SVT is also below a SVT threshold amount, flow moves downward from 508 to 513. At 513, neither VT or SVT data or probabilities are displayed. Instead, an indication may be made, that based on COIs, the results are inconclusive. Alternatively, a probability of a different arrhythmia may be displayed. In either instance, the COIs may be displayed for review by a clinician.

At 514, a treatment is applied based on the COIs determined from 506. Specifically, depending on the information displayed at 508, 512, and 513 a treatment is determined. In one example, information is utilized by a clinician who determines the applied treatment. Alternatively, when a determination at 508 is above a threshold percentage, one or more processors apply the treatment accordingly by actuating the IMD. In one example, if the likelihood that an arrhythmia is a VT is above 95%, the one or more processors may apply a predetermined treatment, including as anti-tachycardia pacing or a shock. This may be done without consultation with a clinician, an appointment, or the like and instead provides immediate treatment and relief, speeding recovery. Alternatively, in another example, if the likelihood that an arrhythmia is an SVT, or a particular type of SVT is above 95%, the one or more processors may command the IMD to not deliver a predetermined treatment, including shock. In this manner the likelihood of an undesired shock may be mitigated or reduced.

In yet another example, the treatment includes scheduling an electrocardiogram (ECG). In this example, the diagnosis data, including the COIs, probabilities of arrythmia type, or the like is recorded in a memory and the treatment circuitry provides information on the display that a consultation for the ECG is recommended. In another example, treatment circuitry in response to a determination that a consultation for an ECG, or other procedure, is recommended, transmits through the transceiver a message to a remote device at a predetermined clinician, hospital, care provider, or the like that scheduling for the ECG, or other procedure, is desired. Specifically, information specific to the patient, may be stored in the memory, including, hut not limited to a work schedule, activities calendar, or the like that may be attached to be utilized by scheduling software or administrators at the hospital, care provider, or the like in order to schedule the ECG. Thus, the speed of treatment for the arrythmia, including further measurements for diagnosis to determine a treatment is provided.

At 516, the one or more processors continue monitoring heart parameters to determine the success of the treatment at 514. For example, if an ATP treatment fails to terminate a diagnosed tachycardia, the number of atrial events will be counted between the last ATP stimulus and first intrinsic ventricular event after ATP is complete. Also, the cycle length of the atrial beats will continue to be measured, and the time from the last ATP stimulus to the first intrinsic ventricular event may be measured. The time from the ATP stimulus to next Atrial event, and the time from the intrinsic Ventricular events to intrinsic Atrial events will be measured. Shortly after ATP delivery is complete, the cycle length of the tachycardia on the RV lead may be measured.

At 518, in response to the determined success of the treatment at 514, an alternative treatment may be applied. In one example, entrainment is provided. Depending upon how the patient, and COIs vary or respond to the ATP treatment, treatment may be varied to prevent implementation of ineffective treatments that may harm or further aggravate the patient or health condition. By utilizing the provided system and method, improved diagnosis and treatment speed are provided while mistreatment and effects thereof are minimized.

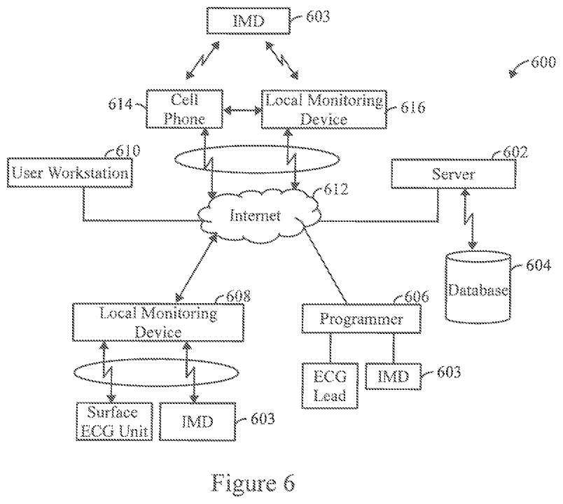

FIG. 6 illustrates a distributed processing system 600 that may also be utilized to communicate information between devices, includes IMDs described in accordance with embodiments herein. The distributed processing system 600 includes a server 602 connected to a database 604, a programmer 606, a local monitoring device 608 and a user workstation 610 electrically connected to a network 612. Any of the processor-based components in FIG. 6 (e.g., workstation 610, cell phone 614, local monitoring device 616, server 602, programmer 606) may perform the processes discussed herein.

The network 612 may provide cloud-based services over the internet, a voice over IP (VoIP) gateway, a local plain old telephone service (POTS), a public switched telephone network (PSTN), a cellular phone-based network, and the like. Alternatively, the communication system may be a local area network (LAN), a medical campus area network (CAN), a metropolitan area network (MAN), or a wide area network (WAM). The communication system serves to provide a network that facilitates the transmission/receipt of data and other information between local and remote devices (relative to a patient). The remote devices include devices at a clinician office or hospital. In this manner, when a probability of a type of arrythmia is determined, a signal may be transmitted to the remote device to schedule an appointment for a follow up procedure.

The server 602 is a computer system that provides services to the other computing devices on the network 612. The server 602 controls the communication of information including cardiac activity signals, cardiac signal waveforms, heart rates, arrythmia data, arrythmia type probabilities, requests to schedule procedures, or the like. The server 602 interfaces with the network 612 to transmit information between the programmer 606, local monitoring devices 608, 616, user workstation 610, cell phone 614 and database 604, including signals that include a probability of an arrythmia type to an external device. The database 604 stores information including cardiac activity signals, cardiac signal waveforms, heart rates, arrythmia data, arrythmia type probabilities, device settings or the like, for a patient population. The information is downloaded into the database 604 via the server 602 or, alternatively, the information is uploaded to the server 602 from the database 604. The programmer 606 may reside in a patient's home, a hospital, or a physician's office. The programmer 606 may wirelessly communicate with the IMD 603 and utilize protocols, including Bluetooth, GSM, infrared wireless LANs, HIPERLAN, 3G, satellite, as well as circuit and packet data protocols, and the like. The programmer 606 is able to obtain cardiac activity signals, cardiac signal waveforms, calculated arrythmia probabilities, atrial and ventricular cycle lengths, or the like from the IMD 603.

The user workstation 610 may be utilized by physician or medical personnel to interface with the network 612 to download cardiac activity data and other information discussed herein, schedule procedures or follow up appointments, or the like from the database 604, from the local monitoring devices 608, 616, from the IMD 603, or otherwise. Once downloaded, the user workstation 610 may process the CA signals in accordance with one or more of the operations described above. The user workstation 610 may upload/push settings (e.g., sensitivity profile parameter settings), IMD instructions, other information and notifications to the cell phone 614, local monitoring devices 608, 616, programmer 606, server 602 and/or IMD 603.

The processes described herein in connection with analyzing cardiac activity signals, determining COIs, for determining probabilities of arrythmia type, determining treatment, scheduling follow up procedures and appointments, or the like may be performed by one or more of the devices illustrated in FIG. 6, including but not limited to the IMD 603, programmer 606, local monitoring devices 608, 616, user workstation 610, cell phone 614, and server 602. The process described herein may similarly be distributed between the devices of FIG. 6.

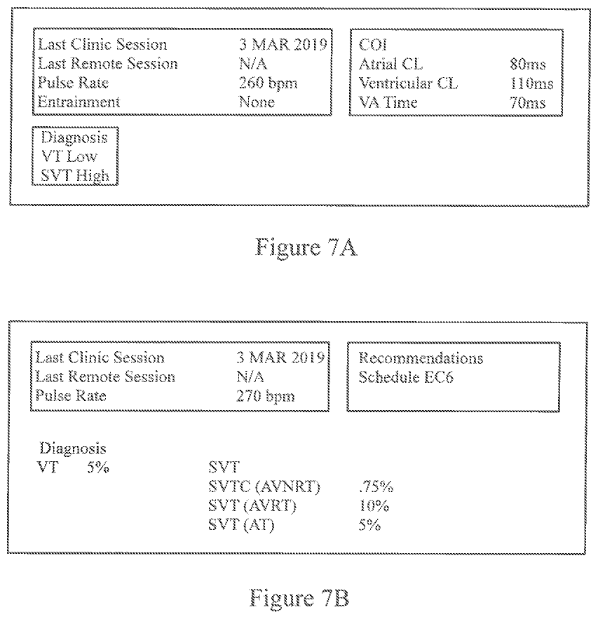

FIGS. 7A and 78 illustrate screenshots of displays in that convey arrythmia data or information to a clinician. Patient information, COI data, recommendations, diagnosis probabilities, or the like may be presented to the clinician to enhance treatment of an arrythmia, and in one example a tachycardia. As illustrated, exact percentages do not have to be provided in order to represent a probability, and instead indictor indicia including “high”, “medium”, “low”, or the like, may be displayed to represent the probability. FIGS. 7A and 78 merely provide example screen shots and additional information may also be included in other example screen shots. Still, the screen shots assist in clinician in diagnosis and treatment of arrhythmia including tachycardia providing more accurate diagnosis, faster care, while reducing undesired treatments and shocks accordingly.

### Closing Statement

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may he in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not Intended to impose numerical requirements on theft objects or order of execution on their acts.

What is claimed is:

1. An Implantable medical device (IMD) for treating an arrhythmia comprising:
electrodes configured to be located proximate to heart tissue of interest, at least a portion of the electrodes configured to deliver therapy, at least a portion of the electrodes configured to sense cardiac activity (CA) signals;
one more processors that, when executing program instructions, are configured to:
deliver antitachycardia pacing (ATP) therapy through the electrodes to the heart tissue of interest in connection with an arrhythmia;
obtain ATP derived CA signals responsive to delivery of the ATP therapy;
determine a characteristic of interest (COI) from the ATP derived CA signals;
calculate a probability of an arrhythmia type based on the COI; and
record the probability of the arrhythmia type.

2. The IMD of claim 1, wherein the ATP derived CA signals are indicative of a response of the heart tissue of interest to the ATP therapy.

3. The IMD of claim 1, wherein the COI includes at least one of atrial cycle length, ventricular cycle length, or ventricular-atrial (VA) interval.

4. The IMD of claim 1, wherein the ATP derived CA signals are obtained during at east a beginning or end of delivering the ATP therapy.

5. The IMD of claim 1, wherein the probability is calculated by comparing the COI detected to historical data of prior COI.

6. The IMD of claim 1, wherein the arrythmia type is one of a terminating ventricular tachycardia (VT), or a supra ventricular tachycardia (SVT).

7. The IMD of claim 1, wherein the calculating the probability includes determining that the arrythmia type is a supra ventricular tachycardia (SVT), and calculating the probability of a type of SVT based on the COI.

8. The IMD of claim 7, wherein the type of SVT is at least one of Atrio-Ventricular Nodal Reentrant Tachycardia (AVNRT), Atrio-Ventricular Reentrant Tachycardia (AVRT), Atrial Tachycardia (AT), Atrial Flutter (AFL), or Atrial Fibrillation (AF).

9. The IMD of claim 1, wherein the one more processors that, when executing program instructions, are configured to: transmit a signal including the probability of the arrhythmia type to an external device.

10. The IMD of claim 1, wherein the one more processors that, when executing program instructions, are configured to: vary pacing of the IMD in response to calculating the probability of the arrythmia type based on the COI.

11. The IMD of claim 1, wherein the one more processors that, when executing program instructions, are configured to: transmit a signal to a remote device related to scheduling a procedure.

12. The IMD of claim 1, wherein the IMD is a leadless pacemaker.

13. A method of treating an arrhythmia with an IMD comprising:
delivering antitachycardia pacing (ATP) therapy through electrodes of the IMD to heart tissue of interest in response to an arrhythmia;
obtaining ATP derived CA signals responsive to delivery of the ATP therapy;
determining a characteristic of interest (COI) from the ATP derived CA signals;
calculating a probability of an arrhythmia type based on the COI; and
recording the probability of the arrhythmia type.

14. The method of claim 13, wherein the ATP derived CA signals are indicative of a response of the heart tissue of interest to the ATP therapy.

15. The method of claim 13, wherein the COI includes at least one of atrial cycle length, ventricular cycle length, or ventricular-atrial (VA) interval.

16. The method of claim 13, wherein the ATP derived CA signals are obtained during at least a beginning or end of delivering the ATP therapy.

17. The method of claim 13, wherein the probability is calculated by comparing the COI to historical data of prior COI.

18. The method of claim 13, wherein the calculating the probability includes determining that the arrythmia type is a supra ventricular tachycardia (SVT), and calculating the probability of a type of SVT based on the COI.

19. The method of claim 13, further comprising: transmitting a signal including the probability of the arrhythmia type calculated to an external device.

20. The method of claim 13, further comprising varying pacing of the IMD in response to calculating the probability of the arrythmia type based on the COI.
